(19) United States
(12) Patent Application Publication
(10) Pub. No.: US 2026/0265921 A1
(43) Pub. Date: Sep. 10, 2026

(54) DETECTION METHOD FOR COPPER FOIL SURFACE

(71) Applicants: National Tsing Hua University, HSINCHU CITY (TW); Chang Chun Plastics Co., Ltd, TAIPEI (TW); Chang Chun PetroChemical Co., Ltd, TAIPEI (TW); DAIREN CHEMICAL CORPORATION, TAIPEI (TW)

(72) Inventors: TZU-CHIEN WEI, HSINCHU CITY (TW); Kala Kannankutty, HSINCHU CITY (TW); Hsin-Yao Ho, Miaoli County (TW); Jui-Chang Chou, Miaoli County (TW)

(21) Appl. No.: 19/011,486

(22) Filed: Jan. 6, 2025

(30) Foreign Application Priority Data
Sep. 11, 2024 (TW) 113134362

(51) Int. Cl. C23C 26/00; G01N 21/3563

(52) U.S. Cl. CPC C23C 26/00; G01N 21/3563

(57) ABSTRACT

A detection method for a copper foil surface includes a black body grafting operation and a surface detection operation. The black body grafting operation includes grafting a black body material onto a first functional group modified on a surface of the copper foil, such that the first functional group is grafted with the black body material. The surface of the black body material is modified with a second functional group, which is capable of grafting with the first functional group. The surface detection operation includes using an infrared thermal imager to detect the surface of the copper foil grafted with the black body material, thereby obtaining an infrared thermal image that displays a distribution state of the first functional group modified on the surface of the copper foil by imaging the black body material.

### CROSS-REFERENCE TO RELATED PATENT APPLICATION

This application claims the benefit of priority to Taiwan Patent Application No. 113134362, filed on Sep. 11, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a detection method, and more particularly to a detection method for a copper foil surface.

### BACKGROUND OF THE DISCLOSURE

With the advancement of high-performance and networked electronic products, the trend toward higher signal frequencies has a significant impact on copper clad laminates (CCLs). CCLs are manufactured by heating and pressing insulating resin substrates and copper foils, which plays a critical role in high-frequency and high-speed applications.

To reduce signal transmission losses, these applications require substrates with low dielectric constants (Low Dk) and low dielectric losses (Low Df). However, these characteristics also weaken the bonding strength between the copper foil and the substrate due to a reduced number of highly polar molecular functional groups.

Moreover, copper clad laminates in high-frequency applications possess low surface roughness. Under high-frequency conditions, currents tend to be concentrated in a thin layer on a conductor surface (e.g., a phenomenon known as the skin effect). However, the roughness of a bonding surface of the copper foil significantly affects signal standing waves and reflections. Therefore, reducing the roughness of the bonding surface of the copper foil is an important measure for mitigating this effect.

To enhance the bonding strength between the copper foil and the insulating resin substrate, a silane coupling agent is used to modify the surface of a bonding side of the copper foil, forming a siloxane cross-linked structure to strengthen chemical bonds. However, challenges exist when modifying with the silane coupling agent. For instance, the silane coupling agent may agglomerate in solvents depending on compatibility between functional groups of the silane coupling agent and properties of the solvent, thereby affecting the uniformity of the modification on the copper foil and the interfacial adhesion. Moreover, the degree of hydrolysis of the silane coupling agent also influences the bonding strength with the copper foil.

Conventionally, there is a lack of effective methods to evaluate the deposition uniformity or the amount of the silane coupling agent modified on the copper foil surface.

Therefore, when the parameters for modifying the copper foil with a silane coupling agent vary, there is no conventional method available for determining whether differences exist in the deposition uniformity or the amount of the silane coupling agent. Variations in deposition amount are generally believed to affect the subsequent bonding strength between the copper foil and the insulating resin substrate.

### SUMMARY OF THE DISCLOSURE

In response to the above-referenced technical inadequacies, the present disclosure provides a detection method for a copper foil surface.

In order to solve the above-mentioned problems, one of the technical aspects adopted by the present disclosure is to provide a detection method for a copper foil surface, which includes providing a copper foil, in which a surface of the copper foil is modified with a first modifier to introduce a first functional group on the surface of the copper foil. The method further includes performing a black body grafting operation and a surface detection operation. The black body grafting operation includes graft-modifying a black body material onto the first functional group of the surface of the copper foil, such that the first functional group is grafted with the black body material, in which a surface of the black body material is modified with a second functional group that is grafted to the first functional group. The surface detection operation includes using an infrared thermal imager to detect the surface of the copper foil grafted with the black body material to obtain an infrared thermal image, in which the infrared thermal image displays a distribution state of the first functional group modified on the surface of the copper foil by imaging the black body material.

In one of the possible or preferred embodiments, the first modifier is an amino silane coupling agent, and the first functional group is an amino silane functional group derived from the amino silane coupling agent. The black body material is at least one selected from the group consisting of carbon black, graphene, and carbon nanotubes; and the second functional group is at least one of a carboxyl group (—COOH) and a hydroxyl group (—OH).

In one of the possible or preferred embodiments, the second functional group is the carboxyl group that forms an electrostatic interaction with an amino terminal of the first functional group, such that the black body material is grafted onto the first functional group on the surface of the copper foil through the second functional group.

In one of the possible or preferred embodiments, the surface of the copper foil has a first emissivity, the black body material has a second emissivity, and the second emissivity is greater than the first emissivity.

In one of the possible or preferred embodiments, the first emissivity is not greater than 0.8, the second emissivity is not less than 0.8, and a ratio of the second emissivity to the first emissivity is not less than 2.

In one of the possible or preferred embodiments, the first emissivity ranges from 0.01 to 0.2, the second emissivity ranges from 0.8 to 1.0, and a ratio of the second emissivity to the first emissivity is not less than 8.

In one of the possible or preferred embodiments, in the black body grafting operation, the copper foil modified with the first functional group is immersed in a dispersion liquid containing the black body material modified with the second functional group, such that the black body material is grafted onto the first functional group through the second functional group.

In one of the possible or preferred embodiments, a concentration of the black body material modified with the second functional group in the dispersion liquid ranges from 0.01 wt % to 0.1 wt %, and an average particle size of the black body material dispersed in the dispersion liquid ranges from 10 nanometers to 200 nanometers.

In one of the possible or preferred embodiments, an immersion time of the copper foil in the dispersion liquid ranges from 10 minutes to 20 minutes.

In one of the possible or preferred embodiments, the concentration of the black body material modified with the second functional group in the dispersion liquid ranges from 0.02 wt % to 0.03 wt %, the average particle size of the black body material dispersed in the dispersion liquid ranges from 10 nanometers to 50 nanometers, and the immersion time of the copper foil in the dispersion liquid ranges from 13 minutes to 17 minutes.

Therefore, in the detection method for the copper foil surface provided by the present disclosure, by virtue of “providing a copper foil, in which a surface of the copper foil is modified with a first modifier to introduce a first functional group on the surface of the copper foil,” “performing a black body grafting operation, which includes graft-modifying a black body material onto the first functional group of the surface of the copper foil, such that the first functional group is grafted with the black body material, in which a surface of the black body material is modified with a second functional group that is grafted to the first functional group,” and “performing a surface detection operation, which includes using an infrared thermal imager to detect the surface of the copper foil grafted with the black body material to obtain an infrared thermal image, in which the infrared thermal image displays a distribution state of the first functional group modified on the surface of the copper foil by imaging the black body material,” the detection method can effectively evaluate a deposition state (e.g., uniformity of modification distribution) of a silane coupling agent on a copper foil surface in a copper-clad laminate manufacturing process.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a detection method for a copper foil surface according to one embodiment of the present disclosure;

FIGS. 2A to 2C are schematic views of steps S110 to S130 of the detection method for the copper foil surface according to one embodiment of the present disclosure;

FIG. 3 is an experimental photograph showing an infrared thermal image of a copper foil surface as detected by an infrared thermal imager according to one embodiment of the present disclosure; and

FIG. 4 is an experimental photograph showing an infrared thermal image of a copper foil surface as detected by an infrared thermal imager according to a comparative example of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Detection Method for Copper Foil Surface

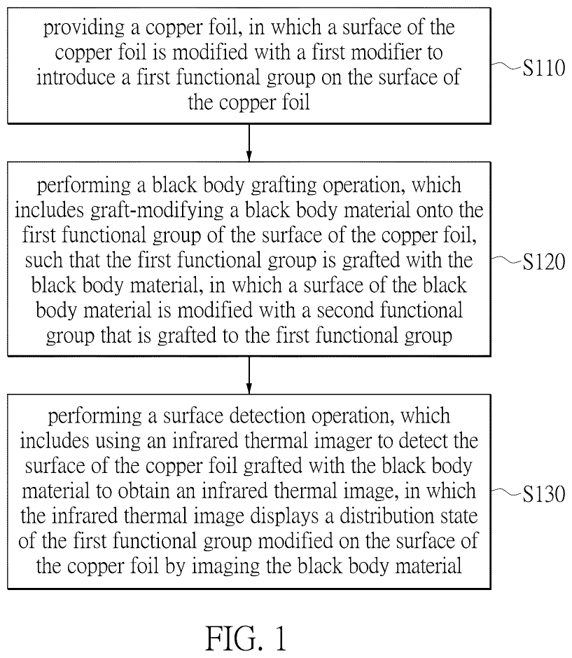

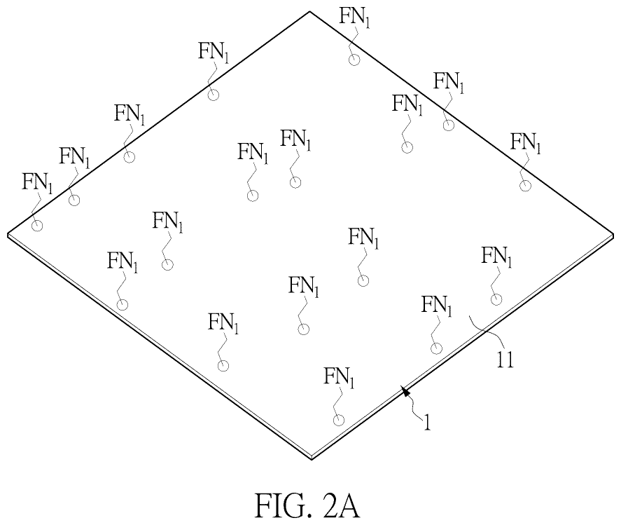

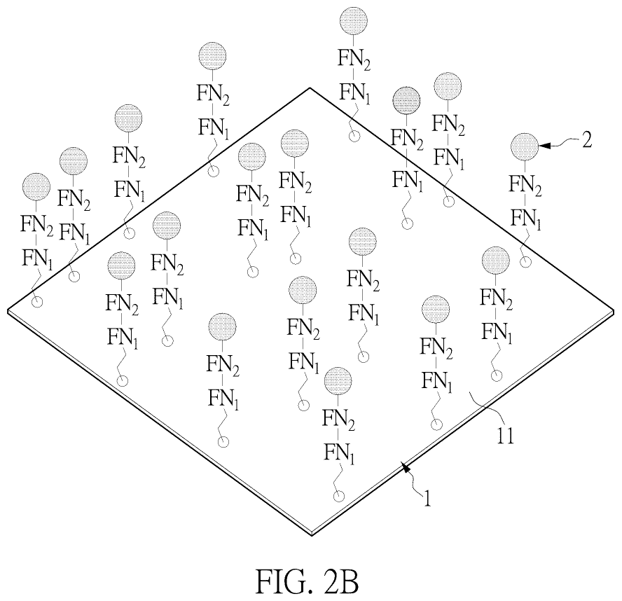

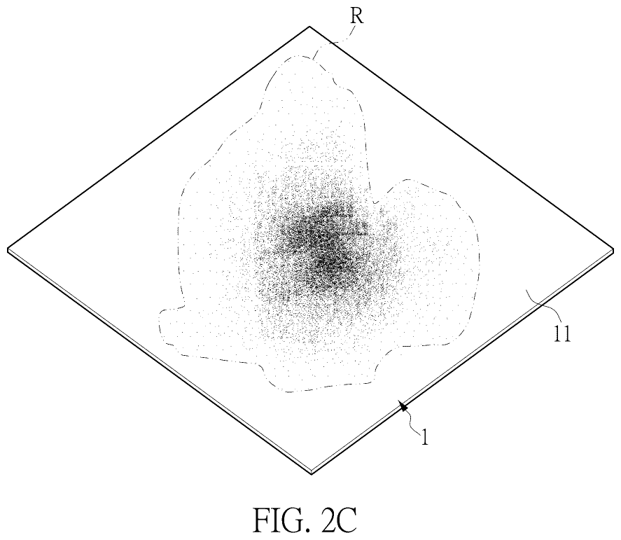

Referring to FIG. 1 and FIGS. 2A to 2C, an embodiment of the present disclosure provides a detection method for a copper foil surface, which is applicable in a technical field of manufacturing copper-clad laminates (CCL) for printed circuit boards.

More specifically, the detection method for the copper foil surface of the embodiment of the present disclosure includes step S110, step S120, and step S130. It should be noted that the sequence and actual operational methods of the steps described in the present embodiment can be adjusted according to practical needs and are not limited to those described in the present embodiment.

As shown in FIG. 1 and FIG. 2A, step S110 includes: providing a copper foil 1, in which a surface 11 (e.g., a bonding surface) of the copper foil 1 is modified with a first modifier, so that the surface 11 of the copper foil 1 is modified with a first functional group FN1. As shown in FIG. 2A, the surface 11 of the copper foil 1 is modified with a plurality of first functional groups FN1. Further, the copper foil 1 is in the form of a thin sheet.

In some embodiments of the present disclosure, the copper foil 1 can be an electrolytic copper foil or a rolled copper foil. For example, the copper foil 1 is an electrolytic copper foil. In addition, a thickness of the copper foil 1 can be, for example, between 3 micrometers and 50 micrometers, and preferably between 5 micrometers and 40 micrometers, but the present disclosure is not limited thereto. In the embodiment of the present disclosure, the first modifier is an amino silane coupling agent. The first functional group FN1 is an amino silane functional group derived from the amino silane coupling agent, and the amino silane functional group has an amino terminal (−NH₂).

For example, the amino silane coupling agent can be 3-[2-(2-aminoethylamino)ethylamino]propyl-trimethoxysilane (ETAS), but the present disclosure is not limited thereto.

The surface 11 of the copper foil 1 can be modified with the first functional group(s) FN1 (i.e., amino silane functional group) by immersing the copper foil 1 in an aqueous solution containing the amino silane coupling agent with a concentration of about 0.1 V/V % to 10 V/V %. It is worth mentioning that the abovementioned surface modification of the copper foil can be a pretreatment in a preparation process of copper-clad laminates (CCL) for printed circuit boards, such as a pretreatment operation performed on the copper foil before laminating the copper foil with an insulating resin substrate.

As shown in FIG. 1 and FIG. 2B, step S120 includes: performing a black body grafting operation, which includes using a black body material 2 (e.g., black body particles) to perform a graft modification reaction on the first functional group FN1 (e.g., amino silane functional group) modified on the surface 11 of the copper foil 1, thereby grafting the black body material 2 onto the first functional group FN1 (e.g., amino terminal), so as to mark the location of the first functional group FN1 by the black body material 2.

In some embodiments of the present disclosure, the black body material 2 is at least one selected from the group consisting of carbon black (e.g., carbon black particles), graphene (e.g., graphene sheets or particles), and carbon nanotubes. For example, the black body material 2 is multi-walled carbon nanotubes (MWCNT), but the present disclosure is not limited thereto.

Furthermore, a surface of the black body material 2 is modified with a second functional group FN2. In some embodiments of the present disclosure, the second functional group FN2 is at least one of a carboxyl group (—COOH) and a hydroxyl group (—OH). More specifically, as shown in FIG. 2B, surfaces of the black body particles are surface-modified with one or more second functional groups FN2 (e.g., carboxyl groups or hydroxyl groups), but the present disclosure is not limited thereto.

For example, the second functional group FN2 is a carboxyl group that can form electrostatic interaction with the amino terminal of the first functional group FN1, so that the black body material 2 can be grafted onto the first functional group FN1 modified on the surface 11 of the copper foil 1 through the second functional group FN2.

More specifically, when the detection method of the embodiment of the present disclosure is applied to the preparation of copper-clad laminates (CCL), the black body grafting operation includes taking out at least a part of the sheet from the copper foil 1 modified with the first functional group FN1 provided in step S110 as a detection sample, and performing graft modification on the first functional group FN1 of the surface 11 of the copper foil 1 (e.g., detection sample) using the black body material 2, thereby grafting the black body material 2 onto the first functional group FN1 of the surface 11 of the copper foil 1 through the second functional group FN2 (e.g., carboxyl group).

In some embodiments of the present disclosure, the surface 11 of the copper foil 1 is a polished surface, which has a first emissivity ε1. The black body material 2 has a second emissivity ε2, and the second emissivity ε2 is greater than the first emissivity ε1. Accordingly, the first functional group FN1 modified on the surface 11 of the copper foil 1 can be accurately targeted by the black body material 2, so as to achieve high resolution under infrared thermal imaging. For example, the first emissivity ε1 is not greater than 0.8, and preferably between 0.01 and 0.2. The second emissivity ε2 is not less than 0.8 and preferably between 0.8 and 1.0. In addition, a ratio of the second emissivity ε2 divided by the first emissivity ε1 (i.e., ε2/ε1) is not less than 2, and preferably not less than 8, so as to effectively enhance resolution.

If the first functional group FN1 modified on the surface 11 of the copper foil 1 is not grafted with the black body material 2 through the second functional group FN2, the first functional group FN1 may have low resolution under infrared thermal imaging, which is difficult to be detected using infrared thermal imaging.

It should be noted that the first emissivity ε1 and the second emissivity ε2 mentioned above refer to the emissivity (ε) measured by an infrared thermometer within a temperature range of 20° C. to 100° C. (e.g., 25° C.), but the present disclosure is not limited thereto.

Furthermore, the copper foil 1, surface-modified with the first functional group FN1, can be immersed in a dispersion liquid containing the black body material 2 (e.g., carbon nanotubes) modified with carboxyl groups in a concentration of about 0.01 wt % to 0.1 wt % and having an average particle size of 10 nanometers to 200 nanometers, so that the black body material 2 is grafted onto the first functional group FN1, which facilitates subsequent detection of the copper foil surface by infrared thermal imaging.

As shown in FIG. 1 and FIG. 2C, step S130 includes: performing a surface detection operation, which includes using an infrared thermal imager (e.g., IR imager or infrared thermal imaging camera) to detect the surface 11 of the copper foil 1 grafted with the black body material 2 to obtain an infrared thermal image R. The infrared thermal image R correspondingly displays a distribution state of the first functional group(s) FN1 (e.g., amino silane functional group) modified on the surface 11 of the copper foil 1 by imaging the black body material 2, thereby assisting in evaluating the modification quality of the silane coupling agent modified on the copper foil, such as the uniformity or concentration of the silane coupling agent modified on the copper foil surface.

For example, the evaluation of uniformity can be conducted by assessing a color temperature distribution state of the infrared thermal image R.

The infrared thermal imager has high detection sensitivity for materials with high emissivity (e.g., the black body material 2) but low detection sensitivity for materials with low emissivity (e.g., copper foil). The black body material 2, grafted with the second functional group FN2 (e.g., carboxyl group), only interacts with the first functional group FN1 (e.g., amino functional group) on the surface 11 of the copper foil 1 and does not interact with areas of the copper foil surface that lack modification by the first functional group FN1.

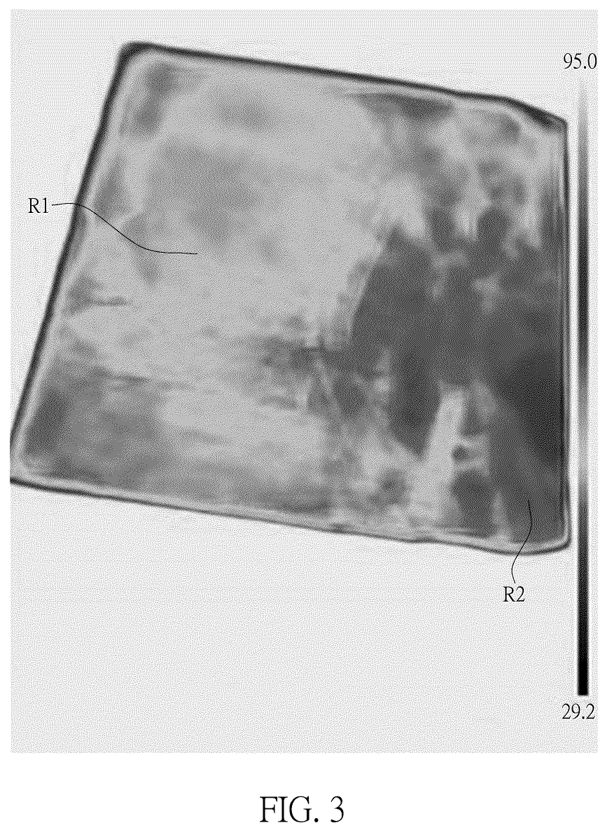

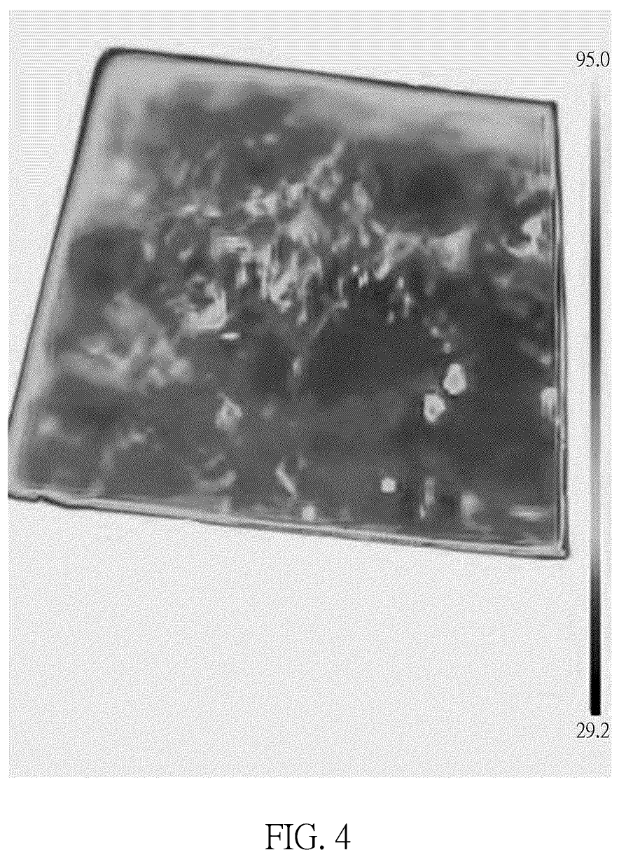

In the amino silane-modified copper foil sample, the black body material 2, grafted with the second functional group FN2, deposits specifically on the amino-functionalized surface, which emits a bright signal in the infrared thermal image, whereas the unmodified copper foil surface appears dim. Relevant experimental images are shown in FIG. 3 and FIG. 4.

FIG. 3 is an experimental photograph of a copper foil surface detected by an infrared thermal imager according to an embodiment of the present disclosure. In the photograph, the color temperatures in the left region (R1) range from 40° C. to 60° C. without significant variation, which indicates a uniform distribution of the amino silane functional groups in the left region (R1). In contrast, the color temperatures in the right region (R2) are below 40° C., which indicates the absence of amino silane functional group on the copper foil surface. Additionally, FIG. 4 shows an experimental photograph of a comparative example of the copper foil surface detected by the infrared thermal imager, where the color temperatures across most (90%) of the copper foil surface are below 40° C., which indicates that the copper foil surface lacks a uniform distribution of amino silane functional groups.

In some embodiments of the present disclosure, a method for evaluating the uniformity of amino silane functional groups modified on a copper foil surface includes extracting a predetermined number of color temperature values per unit area from the infrared thermal image R, and calculating an average value and a standard deviation of the color temperature values. If the average value and the standard deviation fall within certain ranges (e.g., the standard deviation being not greater than 2, and preferably not greater than 0.5), the uniformity of the silane coupling agent modified on the copper foil surface is considered uniform. If the average value and the standard deviation exceed the above range (e.g., the standard deviation being greater than 2), the uniformity of the silane coupling agent modified on the copper foil surface is evaluated as non-uniform.

For example, the average value and the standard deviation falling within specific ranges indicates that the black body material is evenly dispersed within the unit area, which corresponds to the fact that the silane coupling agent is uniformly modified on the surface of the copper foil.

It should be noted that the above description is an illustrative example of the evaluation method, but the present disclosure is not limited thereto.

In conclusion, the detection method for the copper foil surface of the present embodiment involves providing a surface modified copper foil by a silane coupling agent. Before laminating the copper foil with an insulating resin substrate (e.g., CCL lamination), at least part of the copper foil is taken out as a detection sample. Then, a black body material (e.g., carbon black particles, graphene sheets, or carbon nanotubes) is used to graft-modify the silane coupling agent on the copper foil surface. Finally, the infrared thermal imaging is employed to detect the copper foil surface, thereby evaluating the quality of the silane coupling agent modified on the copper foil surface, such as the uniformity of the modification on the copper foil surface. If the evaluation result is uniform, the remaining copper foil material can be transported to the subsequent lamination operation to be laminated with the insulating resin substrate, ensuring the reliability of the subsequent lamination process (e.g., the bonding strength between the copper foil and the insulating resin substrate).

It is worth mentioning that the polished surface of the copper foil has low emissivity, which results in low resolution under infrared thermal imaging (e.g., making it difficult to observe the state of silane modification). The detection method provided by the embodiment of the present disclosure includes grafting a black body material having high-emissivity onto the amino silane functional group modified on the copper foil, achieving targeted marking through the specific interaction between the COOH group on the black body material and the NH₂ group on the silane functional group, thereby reducing the detection time to be less than ten minutes. The detection method provided by the present embodiment is highly feasible, and is capable of improving the process yield of copper-clad laminates (CCL) and reducing production costs.

Furthermore, to effectively enhance the graft modification efficiency of the second functional group FN2 (e.g., carboxyl group) on the black body material 2 to the first functional group FN1 (i.e., amino silane functional group) on the surface 11 of the copper foil 1, and to effectively mark the position of the first functional group FN1 on the surface 11 of the copper foil 1, the embodiment of the present disclosure provides more optimized implementation methods for the preparation of the black body material 2 and the graft modification conditions for the first functional group FN1.

However, the present disclosure is not limited to the following exemplary implementations.

The preparation method of the black body material 2 according to the embodiment of the present disclosure includes: adding 0.1 grams to 0.3 grams, preferably 0.15 grams to 0.25 grams, of a black body material (e.g., multi-walled carbon nanotubes, MWCNT) into a solution formed by mixing sulfuric acid (H₂SO₄) and nitric acid (HNO₃) to form a reaction solution. A volume of sulfuric acid is between 14 milliliters to 28 milliliters (preferably 20 milliliters to 22 milliliters), a volume of nitric acid is between 5 milliliters to 9 milliliters (preferably 6 milliliters to 8 milliliters), and a volume ratio of sulfuric acid to nitric acid is 2 to 4:1 (preferably 2.5 to 3.5:1). The reaction solution is then heated to 60° C. to 80° C. (preferably 65° C. to 75° C.) and treated with ultrasound for 2 hours to 4 hours (preferably 2.5 hours to 3.5 hours), where the ultrasonic frequency can, for example, range between 40 KHz and 45 KHz.

Accordingly, the black body material (i.e., carbon nanotubes) is modified with carboxyl groups to form a carboxyl-modified black body material (e.g., CNT-COOH).

After the reaction is completed, the solid reactant (e.g., the carboxyl-modified black body material) in the reaction solution is separated and washed with deionized water until the filtrate reaches neutral (e.g., pH 7). The solid reactant is then dried, completing the modification of the black body material, and the dried carboxyl-modified black body material is collected for the subsequent detection operation.

The dried carboxyl-modified black body material can be further dispersed in a solvent to form a dispersed liquid (e.g., CNT-COOH dispersed liquid).

In an embodiment of the present disclosure, the solvent is water (H₂O), which provides a more stable dispersion effect for the carboxyl-modified black body material compared to organic solvents (e.g., isopropanol, IPA), with no solid precipitation or phase separation observed after being stored for a period of time (e.g., three days).

In addition, a concentration of the carboxyl-modified black body material in the solvent (e.g., water) ranges from 0.01 wt % to 0.1 wt %, and preferably ranges from 0.020 wt % to 0.030 wt %.

In a preparation method, 38 milligrams of carboxyl-modified black body material is dispersed in 38 milliliters of water, but the present disclosure is not limited thereto.

To improve the dispersion of the carboxyl-modified black body material in the solvent, the dispersed liquid can be treated with ultrasound for 1 to 6 hours (preferably 3 to 5 hours), with an ultrasonic frequency ranging between 40 KHz and 45 KHz.

In terms of particle size, a dispersion particle size of the carboxyl-modified black body material in the solvent ranges from 10 nanometers to 200 nanometers, preferably ranges from 10 nanometers to 50 nanometers, so as to effectively and individually mark each amino silane functional group position on the copper foil surface.

The copper foil with an amino silane-modified surface is then immersed in the dispersed liquid containing the carboxyl-modified black body material to graft the carboxyl-modified black body material onto the amino silane functional groups on the surface of the copper foil. An immersion time of the copper foil in the dispersed liquid ranges from 1 minute to 30 minutes, preferably ranges from 10 minutes to 20 minutes, and more preferably ranges from 13 minutes to 17 minutes. After the immersion process for the copper foil is completed, the copper foil (e.g., CNT-COOH modified Cu-foil) is taken out and dried using an air gun, which is prepared for the subsequent detection.

It is worth mentioning that if the surface of the copper foil is modified with the amino silane functional groups, the surface of the copper foil that has been immersed in the dispersed liquid forms black regions, while areas of the surface of the copper foil without amino silane functional group modification retains the original color of the copper foil.

The above technical solution effectively enhances the graft modification efficiency of the carboxyl group on the black body material to the amino silane functional groups on the copper foil surface, and effectively marks the positions of the amino silane functional groups on the copper foil.

### Beneficial Effects of the Embodiments

In conclusion, in the detection method for the copper foil surface provided by the present disclosure, by virtue of “providing a copper foil, in which a surface of the copper foil is modified with a first modifier to introduce a first functional group on the surface of the copper foil,” “performing a black body grafting operation, which includes graft-modifying a black body material onto the first functional group of the surface of the copper foil, such that the first functional group is grafted with the black body material, in which a surface of the black body material is modified with a second functional group that is grafted to the first functional group,” and “performing a surface detection operation, which includes using an infrared thermal imager to detect the surface of the copper foil grafted with the black body material to obtain an infrared thermal image, in which the infrared thermal image displays a distribution state of the first functional group modified on the surface of the copper foil by imaging the black body material,” the detection method can effectively evaluate a deposition state (e.g., uniformity of modification distribution) of a silane coupling agent on a copper foil surface in a copper-clad laminate manufacturing process.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A detection method for a copper foil surface, comprising:
providing a copper foil, wherein a surface of the copper foil is modified with a first modifier to introduce a first functional group on the surface of the copper foil;
performing a black body grafting operation, which includes graft-modifying a black body material onto the first functional group of the surface of the copper foil, such that the first functional group is grafted with the black body material, wherein a surface of the black body material is modified with a second functional group that is grafted to the first functional group; and
performing a surface detection operation, which includes using an infrared thermal imager to detect the surface of the copper foil grafted with the black body material to obtain an infrared thermal image, wherein the infrared thermal image displays a distribution state of the first functional group modified on the surface of the copper foil by imaging the black body material.

2. The detection method according to claim 1, wherein the first modifier is an amino silane coupling agent, and the first functional group is an amino silane functional group derived from the amino silane coupling agent;
wherein the black body material is at least one selected from the group consisting of carbon black, graphene, and carbon nanotubes; and the second functional group is at least one of a carboxyl group (—COOH) and a hydroxyl group (—OH).

3. The detection method according to claim 2, wherein the second functional group is the carboxyl group that forms an electrostatic interaction with an amino terminal of the first functional group, such that the black body material is grafted onto the first functional group on the surface of the copper foil through the second functional group.

4. The detection method according to claim 1, wherein the surface of the copper foil has a first emissivity, the black body material has a second emissivity, and the second emissivity is greater than the first emissivity.

5. The detection method according to claim 4, wherein the first emissivity is not greater than 0.8, the second emissivity is not less than 0.8, and a ratio of the second emissivity to the first emissivity is not less than 2.

6. The detection method according to claim 4, wherein the first emissivity ranges from 0.01 to 0.2, the second emissivity ranges from 0.8 to 1.0, and a ratio of the second emissivity to the first emissivity is not less than 8.

7. The detection method according to claim 3, wherein, in the black body grafting operation, the copper foil modified with the first functional group is immersed in a dispersion liquid containing the black body material modified with the second functional group, such that the black body material is grafted onto the first functional group through the second functional group.

8. The detection method according to claim 7, wherein a concentration of the black body material modified with the second functional group in the dispersion liquid ranges from 0.01 wt % to 0.1 wt %, and an average particle size of the black body material dispersed in the dispersion liquid ranges from 10 nanometers to 200 nanometers.

9. The detection method according to claim 8, wherein an immersion time of the copper foil in the dispersion liquid ranges from 10 minutes to 20 minutes.

10. The detection method according to claim 9, wherein the concentration of the black body material modified with the second functional group in the dispersion liquid ranges from 0.02 wt % to 0.03 wt %, the average particle size of the black body material dispersed in the dispersion liquid ranges from 10 nanometers to 50 nanometers, and the immersion time of the copper foil in the dispersion liquid ranges from 13 minutes to 17 minutes.
